(19) United States
(12) Patent Application Publication
(10) Pub. No.: US 2026/0000802 A1
(43) Pub. Date: Jan. 1, 2026

(54) STERILIZATION INDICATOR READING APPARATUS AND METHOD

(71) Applicant: Solventum Intellectual Properties Company, Maplewood, MN (US)

(72) Inventor: Peter D. Ludowise, Cottage Grove, MN (US)

(21) Appl. No.: 19/113,154

(22) PCT Filed: Sep. 12, 2023
(86) PCT No.: PCT/IB2023/059044

§ 371 Date: Mar. 19, 2025

Related U.S. Application Data
(60) Provisional application No. 63/376,449, filed on Sep. 21, 2022.

(51) Int. Cl. A61L 2/28; C12Q 1/22

(52) U.S. Cl. CPC A61L 2/28; C12Q 1/22; A61L 2202/14

(57) ABSTRACT

A sterilization indicator reading apparatus is provided. The reading apparatus includes a housing including a top portion defining an aperture therethrough. The aperture is dimensioned to at least partially receive a sterilization indicator therethrough, such that the sterilization indicator is at least partially received within the housing. The reading apparatus further includes a heating coil disposed within the housing. Upon insertion of the sterilization indicator within the housing through the aperture, the heating coil is spaced apart from the sterilization indicator. The heating coil is configured to radiatively heat the sterilization indicator. The reading apparatus further includes an air supply unit disposed within the housing and configured to selectively supply air to the heating coil.

### TECHNICAL FIELD

The present disclosure generally relates to a sterilization indicator reading apparatus and a method for incubating a sterilization indicator.

### BACKGROUND

In a variety of industries, such as the health care industry, but also in other industrial applications, it may be necessary to monitor effectiveness of processes used to sterilize equipment, such as medical devices, instruments, and other disposable and non-disposable articles. A sterilization cycle is generally defined as a process of completely destroying all viable sources of biological activity, such as microorganisms, including structures such as viruses and spores. As a standard practice, hospitals or other institutions include a sterilization indicator with a batch of articles to assay the lethality of the sterilization cycle/process. Both biological and chemical sterilization indicators may be used.

The biological sterilization indicator may include a known quantity of test microorganisms, for example, Geobacillus stearothermophilus (formerly Bacillus stearothermophilus) or Bacillus atrophacus (formerly Bacillus subtilis) spores, which may be many times more resistant to particular sterilization processes than other contaminating organisms. After the exposure of the indicator to the sterilization process, the sources of biological activity (e.g., spores) may be incubated in a liquid nutrient medium to determine an effectiveness of the sterilization process, for example, whether any of the sources survived the sterilization process, with source metabolism and/or growth indicating that the sterilization process may be insufficient to destroy all the sources of biological activity.

Reading apparatuses may be used to read the biological sterilization indicators after the biological sterilization indicators undergo the sterilization process to determine effectiveness of the sterilization process, i.e., whether the sterilization process was able to effectively destroy the test microorganisms of the biological sterilization indicators. However, conventional reading apparatuses may be slow to read the biological sterilization indicators, thereby causing a delay in obtaining a result indicative of the effectiveness of the sterilization process.

### SUMMARY

In a first aspect, the present disclosure provides a sterilization indicator reading apparatus for use with a sterilization indicator having spores and a substance fluorescently responsive to a spore concentration. The sterilization indicator reading apparatus includes a housing including a top portion and a bottom portion opposite the top portion. The top portion defines an aperture therethrough. The aperture is dimensioned to at least partially receive the sterilization indicator therethrough, such that the sterilization indicator is at least partially received within the housing. The sterilization indicator reading apparatus further includes a heating coil disposed within the housing between the top portion and the bottom portion. The heating coil is at least partially aligned with the aperture and defines an interior volume therein. Upon insertion of the sterilization indicator within the housing through the aperture, the sterilization indicator is at least partially received in the interior volume of the heating coil. The heating coil is dimensioned, such that the heating coil is spaced apart from the sterilization indicator when the sterilization indicator is at least partially received in the interior volume of the heating coil and the heating coil is configured to radiatively heat the sterilization indicator. The heating coil is configured to be switched between an on state and an off state. In the on state, the heating coil is heated. In the off state, the heating coil is not heated. The sterilization indicator reading apparatus further includes an air supply unit disposed within the housing and configured to selectively supply air to the heating coil. The air supply unit is configured to be switched between an active state and an inactive state. In the active state, the air supply unit supplies air to the heating coil. In the inactive state, the air supply unit does not supply air to the heating coil.

In a second aspect, the present disclosure provides a sterilization indicator reading apparatus for use with a sterilization indicator having spores and a substance fluorescently responsive to a spore concentration. The sterilization indicator reading apparatus includes a housing including a top portion and a bottom portion opposite the top portion. The top portion defines an aperture therethrough. The aperture is dimensioned to at least partially receive the sterilization indicator therethrough, such that the sterilization indicator is at least partially received within the housing. The sterilization indicator reading apparatus further includes a heating coil disposed within the housing between the top portion and the bottom portion. Upon insertion of the sterilization indicator within the housing through the aperture, the sterilization indicator is disposed proximal to and spaced apart from the heating coil, such that the heating coil is configured to radiatively heat the sterilization indicator. The heating coil is configured to be switched between an on state and an off state. In the on state, the heating coil is heated. In the off state, the heating coil is not heated. The sterilization indicator reading apparatus further includes an air supply unit disposed within the housing and configured to selectively supply air to the heating coil. The air supply unit is configured to be switched between an active state and an inactive state. In the active state, the air supply unit supplies air to the heating coil. In the inactive state, the air supply unit does not supply air to the heating coil. The sterilization indicator reading apparatus further includes a processor communicably coupled to the heating coil and the air supply unit. The processor is configured to control the heating coil and the air supply unit, such that the heating coil is in the on state and a temperature of the heating coil rises to a first temperature in a first time period. The air supply unit is maintained in the inactive state during the first time period. The processor is further configured to control the heating coil and the air supply unit, such that the heating coil is in the on state and the temperature of the heating coil is maintained between a second temperature and a third temperature during a second time period subsequent to the first time period. The air supply unit is maintained in the active state during the second time period in order to maintain the temperature of the heating coil between the second temperature and the third temperature. The processor is further configured to control the heating coil and the air supply unit, such that the heating coil is maintained in the off state during a third time period subsequent to the second time period. The air supply unit is maintained in the active state during the third time period in order to cool the heating coil.

In a third aspect, the present disclosure provides a method for incubating a sterilization indicator having spores and a substance fluorescently responsive to a spore concentration. The method includes inserting the sterilization indicator at least partially through an aperture of a housing, such that the sterilization indicator is disposed proximal to and spaced apart from a heating coil disposed within the housing. The heating coil is configured to radiatively heat the sterilization indicator. The method further includes raising a temperature of the heating coil to a first temperature in a first time period by controlling, via a processor, power supply to the heating coil and the air supply unit during the first time period, such that the heating coil is an on state and the air supply unit is in an inactive state during first time period. In the on state, the heating coil is heated. In the inactive state, the air supply unit does not supply air to the heating coil. The method further includes maintaining the temperature of the heating coil between a second temperature and a third temperature during a second time period subsequent to the first time period by controlling, via the processor, power supply to the heating coil and the air supply unit during the second time period, such that the heating coil is maintained in the on state and the air supply unit is maintained in an active state during the second time period. In the active state, the air supply unit supplies air to the sterilization indicator. The method further includes reducing the temperature of the heating coil during a third time period subsequent to the second time period by controlling, via the processor, power supply to the heating coil and the air supply unit, such that the heating coil is maintained in an off state and the air supply unit is maintained in the active state during the third time period. In the off state, the heating coil is not heated.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a schematic front perspective view of an exemplary sterilization indicator;

FIG. 2 is a schematic front perspective view of a system including a sterilization indicator reading apparatus with the sterilization indicator received therein according to an embodiment of the present disclosure;

FIG. 3 is a schematic cross-sectional view of the sterilization indicator reading apparatus according to an embodiment of the present disclosure;

FIG. 4 is a graph depicting a variation of a temperature of a heating coil of the sterilization indicator reading apparatus with respect to time according to an embodiment of the present disclosure;

FIG. 5A is schematic cross-sectional view of a portion of a sterilization indicator reading apparatus according to another embodiment of the present disclosure;

FIG. 5B is schematic cross-sectional view of a portion of a sterilization indicator reading apparatus according to another embodiment of the present disclosure;

FIG. 6 is a flowchart depicting various steps of a method according to an embodiment of the present disclosure;

FIG. 7 is an exemplary graph depicting a variation of a temperature of a heater coil with respect to time and a variation of a temperature of a substance of the sterilization indicator due to the variation of the temperature of the heater coil with respect to time; and

FIG. 8 is an exemplary graph depicting a variation of a temperature of a heater coil with respect to time and a variation of a temperature of a substance of the sterilization indicator due to the variation of the temperature of the heater coil with respect to time.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “similar” to a second material, at least 90 weight % of the first and second materials are identical and any variation between the first and second materials comprises less than about 10 weight % of each of the first and second materials.

As used herein, “at least one of A and B” should be understood to mean “only A, only B, or both A and B”.

As used herein, the term “processor” refers a computing device that couples to one or more other devices/circuits, e.g., switching circuits, etc., and which may be configured to communicate with, e.g., to control, such devices/circuits. The processor may include any device that performs logic operations. A processor may include a general processor, a central processing unit, an application specific integrated circuit (ASIC), a digital signal processor, a field programmable gate array (FPGA), a digital circuit, an analog circuit, a microcontroller, any other type of controller, or any combination thereof.

As used herein, the term “communicably coupled” refers to direct coupling between components and/or indirect coupling between components via one or more intervening components. Such components and intervening components may include, but are not limited to, junctions, communication paths, components, circuit elements, circuits, functional blocks, and/or devices. As an example of indirect coupling, a signal conveyed from a first component to a second component may be modified by one or more intervening components by modifying the form, nature, or format of information in a signal, while one or more elements of the information in the signal are nevertheless conveyed in a manner than can be recognized by the second component.

As used herein, the term “sterilization” generally refers to a process of eliminating all bacteria and other living organisms from the surfaces of instruments, medical devices, implants, and other articles used in sterile surgical procedures. A conventional thermal sterilization process uses steam under pressure. Low-temperature chemical sterilization processes use ethylene oxide, hydrogen peroxide, hydrogen peroxide/plasma, or peracetic acid in liquid or vapor form as the sterilant, as well as gamma irradiation and electron beam sterilization.

As used herein, the term “sterilizer” refers to a system or an apparatus that can carry out a sterilization cycle, i.e., a process of completely destroying all viable sources of biological activity, such as microorganisms, including structures such as viruses and spores.

As used herein, the term “result” refers to an outcome indicative of an effectiveness of a sterilization cycle that can be determined by a sterilization indicator reading apparatus by reading a sterilization indicator that has undergone the sterilization cycle. The result may be positive or negative. A positive result refers to an unsuccessful sterilization of the sterilization indicator after undergoing the sterilization process. The positive result may be determined by the sterilization indicator reading apparatus upon detection of a presence of biological activity (for example, by detection of unsterilized microorganisms) in the sterilization indicator after undergoing the sterilization process. A negative result refers to a successful sterilization of the sterilization indicator after undergoing the sterilization process. The negative result may be determined by the sterilization indicator reading apparatus when the presence of biological activity is not detected in the sterilization indicator after undergoing the sterilization process. The negative result is indicative of effective sterilization. In other words, the negative result indicates that microorganisms in the sterilization indicator are effectively killed during the sterilization process.

Various sterilization indicators may be used for different sterilization processes using steam, hydrogen peroxide gas, ethylene oxide, and the like. The sterilization indicators may carry a biological agent. The sterilization indicators are typically placed in a test package within a load containing articles to be sterilized. The sterilization indicators may indicate successful sterilization when the biological agent has been killed. The biological agent carried by the sterilization indicators is typically a test organism which is many times more resistant to the sterilization process than most organisms that are present due to natural contamination. The biological agent may include microorganisms, such as endospores, bacterial spores, or the like.

Reading apparatuses may be used to read the biological sterilization indicators after the biological sterilization indicators undergo the sterilization process to determine effectiveness of the sterilization process, i.e., whether the sterilization process was able to effectively destroy the test microorganisms of the biological sterilization indicators. However, conventional reading apparatuses may be slow to read the biological sterilization indicators, thereby causing a delay in obtaining a result indicative of the effectiveness of the sterilization process. Conventional reading apparatuses may utilize large heater blocks that require substantial warm-up time and additional energy in heating thereof.

The present disclosure provides a sterilization indicator reading apparatus for use with a sterilization indicator having spores and a substance fluorescently responsive to a spore concentration. The sterilization indicator reading apparatus includes a housing including a top portion, and a bottom portion opposite the top portion. The top portion defines an aperture therethrough. The aperture is dimensioned to at least partially receive the sterilization indicator therethrough, such that the sterilization indicator is at least partially received within the housing. The sterilization indicator reading apparatus further includes a heating coil disposed within the housing between the top portion and the bottom portion. The heating coil is at least partially aligned with the aperture and defines an interior volume therein. Upon insertion of the sterilization indicator within the housing through the aperture, the sterilization indicator is at least partially received in the interior volume of the heating coil. The heating coil is dimensioned, such that the heating coil is spaced apart from the sterilization indicator when the sterilization indicator is at least partially received in the interior volume of the heating coil and the heating coil is configured to radiatively heat the sterilization indicator. The heating coil is configured to be switched between an on state and an off state. In the on state, the heating coil is heated. In the off state, the heating coil is not heated. The sterilization indicator reading apparatus further includes an air supply unit disposed within the housing and configured to selectively supply air to the heating coil. The air supply unit is configured to be switched between an active state and an inactive state. In the active state, the air supply unit supplies air to the heating coil. In the inactive state, the air supply unit does not supply air to the heating coil.

The sterilization indicator reading apparatus of the present disclosure may be used to rapidly and accurately read the sterilization indicators that have undergone a sterilization process to determine an efficacy/effectiveness of the sterilization process. In other words, the sterilization indicator reading apparatus of the present disclosure may reduce a time taken to obtain a result (also known as TTR (time to result)) indicative of the effectiveness of the sterilization process.

The heating coil may be quickly heated in an “on-demand” mode, as compared to conventional reading apparatuses that may utilize large heater blocks requiring substantial warm-up time and additional energy in heating thereof. The heating coil and/or the air supply unit may be controlled by using servo control to rapidly heat the sterilization indicator from room temperature to its optimal temperature, and maintain the optimal temperature throughout incubation, thereby decreasing the time to result of the sterilization indicator reading apparatus.

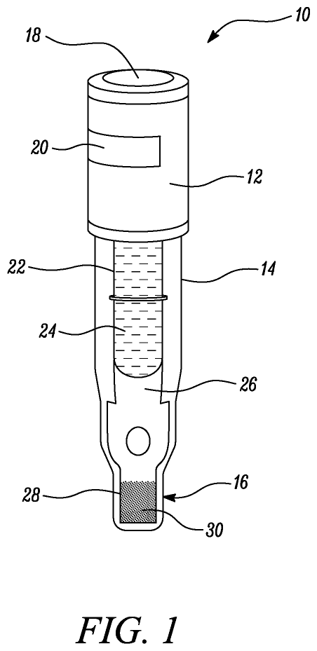

Referring now to the Figures, FIG. 1 illustrates a schematic front perspective view of a sterilization indicator 10. The sterilization indicator 10 is preferably a biological sterilization indicator (e.g., a self-contained biological sterilization indicator). Examples of the biological sterilization indicator are known and are manufactured by companies such as 3M under the trade designation ATTEST, Steris (Mentor, OH) under the trade designation Verify, and Terragene (Argentina).

The sterilization indicator 10 illustrated in FIG. 1 includes a cap 12, an outer vial 14, and a growth chamber 16. The sterilization indicator 10 illustrated in FIG. 1 further includes a process indicator label 18 and an information label 20 disposed on (e.g., adhered to) the cap 12.

The process indicator label 18 may indicate whether the sterilization indicator 10 has been exposed to a sterilization process. For example, the process indicator label 18 may be configured to undergo a color change when exposed to the sterilization process. Therefore, the exposure of the sterilization indicator 10 to the sterilization process may be confirmed by observing the color change in the process indicator label 18. In some examples, the color change of the process indicator label 18 from light pink to brown may indicate that the sterilization indicator 10 has been exposed to the sterilization process.

The information label 20 may include information related to the sterilization indicator 10, such as a vial number, an experiment number, a sterilization process name, or any other information.

In the illustrated example of FIG. 1, the sterilization indicator 10 further includes a media ampoule 22 disposed within the outer vial 14 and having a substance 24, and a spore carrier 28 disposed within the growth chamber 16 and having spores 30. In other words, in the illustrated example of FIG. 1, the sterilization indicator 10 has the substance 24 and the spores 30. The substance 24 is fluorescently responsive to a spore viability (i.e., an ability of the spores 30 to survive a sterilization cycle). In other words, the substance 24 may be fluorescently responsive to a viable spore concentration (i.e., a concentration of the spores 30 that survive the sterilization cycle). Further, a reaction between the spores 30 and the substance 24 may cause the substance 24 to emit fluorescence upon absorbing electromagnetic radiation (e.g., ultraviolet light).

The spores 30 may be selected according to the sterilization process used. For example, for a steam sterilization process. Geobacillus stearothermophilus or Bacillus stearothermophilus may be used. For an ethylene oxide sterilization process. Bacillus atrophacus (formerly Bacillus subtilis) may be used. The spores 30 may be resistant to the sterilization process. The spores 30 may include, but are not limited to, Geobacillus stearothermophilus, Bacillus stearothermophilus, Bacillus subtilis, Bacillus atrophacus, Bacillus megaterium, Bacillus coagulans, Clostridium sporogenes, Bacillus pumilus, or combinations thereof.

The substance 24 (preferably liquid) may be a nutrient medium and can generally be selected to induce germination and initial outgrowth of the spores 30, if viable. The substance 24 may include one or more sugars, including, but not limited to, glucose, fructose, cellobiose, or a combination thereof. The substance 24 may also include a salt, including, but not limited to, potassium chloride, calcium chloride, or a combination thereof. In some embodiments, the substance 24 may further include an amino acid, including, but not limited to, methionine, phenylalanine, tryptophan, and the like.

In one example, the sterilization indicator 10 may include an α-glucosidase enzyme system, which is generated naturally within growing cells of Geobacillus stearothermophilus. The α-glucosidase in its active state may be detected by measuring a fluorescence produced by the enzymatic hydrolysis of the substance 24 (e.g., a non-fluorescent substrate, and 4-methylumbelliferyl-a-D-glucoside (MUG)).

Enzymes and substrates that may be suitable for use in the sterilization indicator 10 are identified in U.S Pat. Nos. 5,073,488 (Matner et al.), 5,418,167 (Matner et al.), and 5,223,401 (Foltz et al.), which are incorporated herein by reference for all they disclose.

The sterilization indicator 10 may have an internal volume of no greater than 0.8 milliliters (mL), no greater than 0.7 mL, no greater than 0.6 mL, no greater than 0.5 mL, no greater than 0.4 mL, no greater than 0.3 mL, or no greater than 0.2 mL. In some examples, the substance 24 may have a volume of about 0.6 mL.

The sterilization indicator 10 may undergo a sterilization cycle/process of a sterilizer. The spores 30 are responsive to an environmental condition in the sterilizer. For example, the environmental condition in the sterilizer may partially or completely destroy biological activity of the spores 30. The environmental condition in the sterilizer may correspond to any one of the physical, gaseous, and liquid sterilization processes. In one example, the environmental condition may include a presence of pressurized steam. In another example, the environmental condition may include presence of any one of vaporized hydrogen peroxide and ethylene oxide.

The media ampoule 22 may be frangible and may be separated from the spore carrier 28. That is, the substance 24 may not be in fluid communication with the spores 30 prior to the media ampoule 22 being fractured, punctured, pierced, crushed, cracked, or the like. To allow mixing of the substance 24 with the spores 30, the outer vial 14 may further include an ampoule crusher 26 disposed between the media ampoule 22 and the spore carrier 28. The media ampoule 22 may be fractured, punctured, pierced, crushed, cracked, or the like via the ampoule crusher 26 by pressing the cap 12 towards the spore carrier 28 with a suitable force. Such a process of mixing of the substance 24 with the spores 30 may be referred to as an activation of the sterilization indicator 10. After undergoing the sterilization cycle/process of the sterilizer, the spores 30 can be exposed to the substance 24 to propagate by the activation of the sterilization indicator 10.

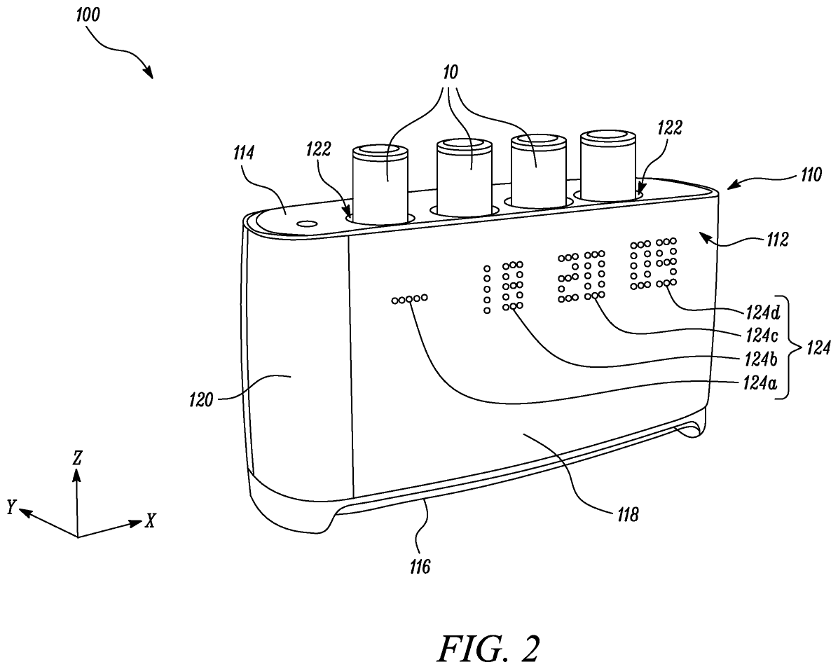

FIG. 2 illustrates a schematic front perspective view of a system 100 including the sterilization indicator 10 and a sterilization indicator reading apparatus 110 (hereinafter referred to as “the reading apparatus 110”) according to an embodiment of the present disclosure. The reading apparatus 110 is for use with the sterilization indicator 10.

The reading apparatus 110 defines mutually orthogonal X, Y, and Z-axes. The X and Y-axes are in-plane axes of the reading apparatus 110, while the Z-axis is a transverse axis disposed along a height of the reading apparatus 110. In other words, the X and Y-axes are disposed along a plane of the reading apparatus 110, while the Z-axis is perpendicular to the plane of the reading apparatus 110.

The reading apparatus 110 includes a housing 112 including a top portion 114 and a bottom portion 116 opposite the top portion 114. The housing 112 may be compact, and may have an internal volume of no greater than 0.5 liter (L), no greater than 0.4 L, no greater than 0.3 L, or no greater than 0.2 L. The housing 112 may therefore be compact while being suitable for use with multiple sterilization indicators 10 simultaneously.

The housing 112 is shown in FIG. 2 as being mostly rectangular, when viewed from a top to bottom direction (i.e., a direction along the Z-axis), and has a rounded rectangular or an ellipsoidal cross-section. The housing 112 may have a major side portion 118 formed along the X-axis and a minor side portion 120 formed along the Y-axis. The top portion 114 of the housing 112 may be flush with an edge of the major side portion 118 but is shown extending above the plane of the major side portion 118 in FIG. 2.

As discussed above, the housing 112 has a rounded rectangular cross-section when viewed from the top to bottom direction. In other words, the housing 112 has two straight edges and two curved edges. The two straight edges may substantially extend along the X-axis, and the two curved edges may partially extend along the Y-axis.

The top portion 114 defines an aperture 122 therethrough. The aperture 122 is dimensioned to at least partially receive the sterilization indicator 10 therethrough, such that the sterilization indicator 10 is at least partially received within the housing 112.

The sterilization indicator 10 may be activated prior to insertion within the housing 112 through the aperture 122. Alternatively, the housing 112 may activate the sterilization indicator 10 upon insertion thereof through the aperture 122. The housing 112 and the sterilization indicator 10 may include one or more features that allow the sterilization indicator 10 to be keyed relative to the housing 112, such as a shelf, a protrusion, or a body shape. As discussed above, the sterilization indicator 10 has the spores 30 (shown in FIG. 1) and the substance 24 (shown in FIG. 1) fluorescently responsive to the spore viability (i.e., the ability of the spores 30 to survive the sterilization cycle). The spores 30 are responsive to the environmental condition in the sterilizer (e.g., the environmental condition in the sterilizer may partially or completely destroy biological activity of the spores 30).

In the illustrated embodiment of FIG. 2, the aperture 122 includes a plurality of apertures 122 spaced apart from each other. Further, in the illustrated embodiment of FIG. 2, a plurality of sterilization indicators 10 is received within corresponding apertures 122 from the plurality of apertures 122. In other words, the reading apparatus 110 may receive the plurality of sterilization indicators 10 through the corresponding apertures 122. Specifically, in the illustrated embodiment of FIG. 2, the plurality of apertures 122 includes four apertures 122 arranged in a linear configuration along the X-axis. However, the plurality of apertures 122 may include any number of the apertures 122 arranged in any suitable configuration.

In the illustrated embodiment of FIG. 2, the reading apparatus 110 further includes a display 124. The display 124 may visually communicate information to a user, e.g., minutes remaining, sterilization indicator pass/fail, or combinations thereof, for each aperture 122 from the plurality of apertures 122. Each aperture 122 may have its own display 124 independent from another display 124 for another aperture 122. As shown in FIG. 2, the display 124 may include a plurality of displays 124a, 124b, 124c, 124d with one display 124 per aperture 122. For example, the displays 124a, 124d may be outer arrays of display elements corresponding to outer apertures 122 from the plurality of apertures 122 (closest to edges of the housing 112). The displays 124b, 124c may be inner arrays of the display elements corresponding to inner apertures 122 from the plurality of apertures 122. In at least one embodiment, a thickness of the major side portion 118 may be greater proximal to the inner apertures 122 than the outer apertures 122. Further, the displays 124b, 124c may be brighter than the displays 124a, 124d.

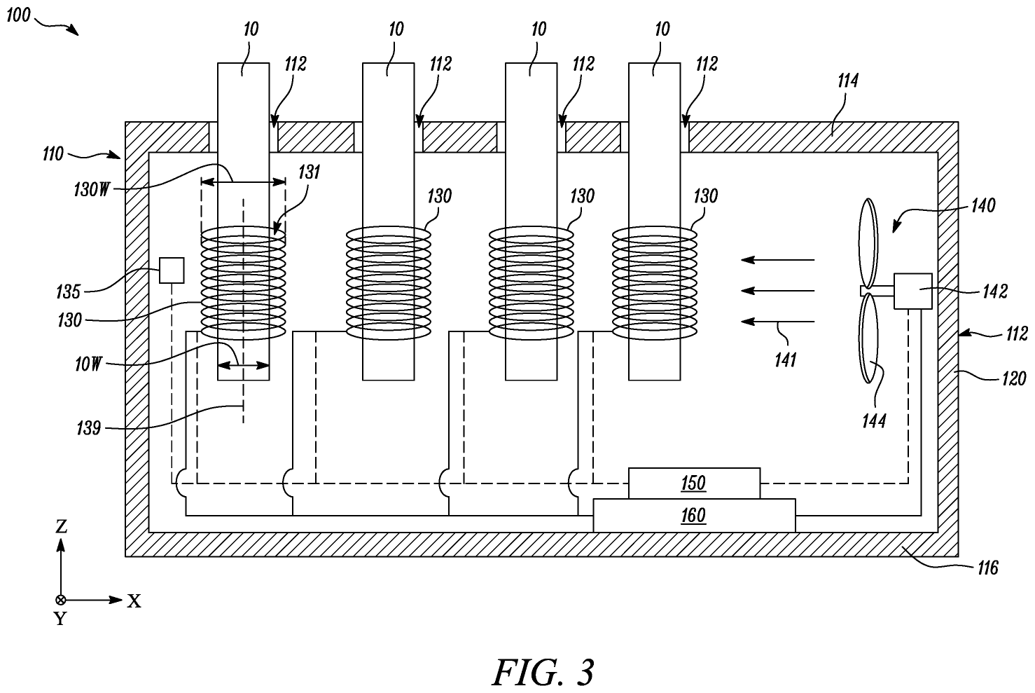

FIG. 3 illustrates a schematic cross-sectional view of the reading apparatus 110.

The reading apparatus 110 further includes a heating coil 130 disposed within the housing 112 between the top portion 114 and the bottom portion 116. Upon insertion of the sterilization indicator 10 within the housing 112 through the aperture 122, the sterilization indicator 10 is disposed proximal to and spaced apart from the heating coil 130, such that the heating coil 130 is configured to radiatively heat the sterilization indicator 10. In other words, the heating coil 130 is positioned within the housing 112, such that the sterilization indicator 10 does not contact the heating coil 130 when inserted within the housing 112 through the aperture 122. The heating coil 130 therefore does not conductively heat the sterilization indicator 10. In contrast, the heating coil 130 is configured to radiatively heat the sterilization indicator 10.

The heating coil 130 may include any suitable material that can be heated in order to radiatively heat the sterilization indicator 10. The material of the heating coil 130 may be stable at high temperatures. The heating coil 130 may include, for example, metal alloys, such as iron-chromium-aluminum (FeCrAl), copper-nickel (CuNi), copper-manganese (CuMn), and the like. Preferably, the heating coil 130 includes nickel-chromium (NiCr). Nickel-chromium has corrosion resistant properties at high temperatures, a high melting point (about 1,400° C.), and a high resistivity. Such properties may make nickel-chromium preferable for selection as the material of the heating coil 130.

In the illustrated embodiment of FIG. 3, the heating coil 130 is at least partially aligned with the aperture 122. The heating coil 130 may be wound about a coil axis 139. The coil axis 139 may be at least partially aligned with the aperture 122. In some embodiments, the coil axis 139 may be substantially parallel to the Z-axis. Upon insertion of the sterilization indicator 10 within the housing 112, a longitudinal axis of the sterilization indicator 10 may be substantially aligned with the coil axis 139.

Furthermore, the heating coil 130 defines an interior volume 131 therein. Upon insertion of the sterilization indicator 10 within the housing 112 through the aperture 122, the sterilization indicator 10 may be at least partially received in the interior volume 131 of the heating coil 130. The heating coil 130 may be dimensioned, such that the heating coil 130 is spaced apart from the sterilization indicator 10 when the sterilization indicator 10 is at least partially received in the interior volume 131 of the heating coil 130 and the heating coil 130 is configured to radiatively heat the sterilization indicator 10. A width 130W of the heating coil 130 may be greater than a maximum width 10W of a portion of the sterilization indicator 10 that is received within the interior volume 131. Therefore, the sterilization indicator 10 may be spaced apart from the heating coil 130 and may not contact the heating coil 130.

The heating coil 130 is configured to be switched between an on state and an off state. In the on state, the heating coil 130 is heated. The on state of the heating coil 130 may refer to a state in which power is supplied to the heating coil 130, such that the heating coil 130 heats up. For example, in the on state of the heating coil 130, electrical power may be supplied to the heating coil 130 resulting in a rise in a temperature of the heating coil 130 due to resistive heating.

In the off state, the heating coil 130 is not heated. The off state of the heating coil 130 may refer to a state in which power supply to the heating coil 130 is discontinued. Therefore, in the off state, the temperature of the heating coil 130 may decrease or be at an ambient temperature within the housing 112.

In the illustrated embodiment of FIG. 3, the heating coil 130 includes a plurality of heating coils 130 corresponding to the plurality of apertures 122. The plurality of apertures 122 and the plurality of heating coils 130 are configured to removably receive a corresponding plurality of sterilization indicators 10. As shown in FIG. 3, each aperture 122 and a corresponding heating coil 130 may removably receive a sterilization indicator 10. In some embodiments, the plurality of heating coils 130 corresponding to the plurality of apertures 122 may be electrically connected to each other in series.

While not illustrated, the plurality of heating coils 130 may be thermally isolated from each other. For example, the reading apparatus 110 may further include an insulating material disposed between adjacent heating coils 130 from the plurality of heating coils 130. In such examples, the reading apparatus 110 may simultaneously incubate the plurality of sterilization indicators 10. Further, the reading apparatus 110 may simultaneously incubate the plurality of sterilization indicators 10 at different temperatures from each other.

The reading apparatus 110 further includes an air supply unit 140 disposed within the housing 112. The air supply unit 140 is configured to selectively supply air 141 (shown by arrows in FIG. 3) to the heating coil 130.

In the illustrated embodiment of FIG. 3, the air supply unit 140 includes at least one fan 144. In some other embodiments, the air supply unit 140 includes at least one blower (not shown). The air supply unit 140 may further include a motor 142 coupled to the at least one fan 144. The motor 142 may be controlled to modulate air 141 supplied by the at least one fan 144. In some embodiments, the motor 142 may be a variable speed motor.

The air supply unit 140 is configured to be switched between an active state and an inactive state. In the active state, the air supply unit 140 supplies air 141 to the heating coil 130. The active state of the air supply unit 140 may refer to a state in which power is supplied to the air supply unit 140, such that the air supply unit 140 supplies air 141 to the heating coil 130. Specifically, in the active state, electric power is suppled to drive the motor 142.

In the inactive state, the air supply unit 140 does not supply air 141 to the heating coil 130. The inactive state of the air supply unit 140 may refer to a state in which power supply to the air supply unit 140 is discontinued. Specifically, in the inactive state, electric power is not supplied to the motor 142, such that the motor 142 is idle. Therefore, in the inactive state, the air supply unit 140 may stop supplying air 141 to the heating coil 130.

In the illustrated embodiment of FIG. 3, the air supply unit 140 is configured to supply air 141 to the plurality of heating coils 130. In some other embodiments, the at least one fan 144 may include a plurality of fans 144 corresponding to the plurality of heating coils 130. Each fan 144 from the plurality of fans 144 may be configured to supply air 141 to the corresponding heating coil 130 from the plurality of heating coils 130. Each fan 144 may be individually controlled based on desired application attributes. This configuration may allow the reading apparatus 110 to simultaneously incubate the plurality of sterilization indicators 10 at different temperatures from each other.

As will be described in greater detail below, the heating coil 130 and the air supply unit 140 may be controlled together to control a temperature of the substance 24 (shown in FIG. 1) of the sterilization indicator 10. Specifically, the heating coil 130 and the air supply unit 140 may be controlled in a closed loop control system, thereby enabling precise control of the temperature of the substance 24 of the sterilization indicator 10.

The reading apparatus 110 may further include a processor 150 (depicted schematically by a block in FIG. 3). The processor 150 may include one or more microprocessors, microcontrollers, digital signal processors (DSPs), application specific integrated circuits (ASICs), field programmable gate arrays (FPGAs), and the like. The processor 150 may be communicably coupled to the heating coil 130 and the air supply unit 140. The processor 150 may be configured to control the heating coil 130 and the air supply unit 140. Specifically, the processor 150 may selectively bring the heating coil 130 to the on state or the off state. The processor 150 may further control the temperature of the heating coil 130 in the on state. Moreover, the processor 150 may selectively bring the air supply unit 140 to the active state or the inactive state. The processor 150 may further control air 141 supplied by the air supply unit 140.

The reading apparatus 110 may further include a power supply 160 communicably coupled to the processor 150. The power supply 160 may be electrically connected to the heating coil 130 and the air supply unit 140. The power supply 160 may be configured to selectively supply power (i.e., electrical power) to the heating coil 130 and the air supply unit 140. The power supply 160 may be adjustable. That is, a magnitude of the power supplied by the power supply 160 may be adjusted and varied based on desired requirements. The power supply 160 may be a variable DC power supply or a variable AC power supply. In some embodiments, the power supply 160 may include a variac.

In the on state of the heating coil 130, the processor 150 may control the power supply 160 to supply power to the heating coil 130. It may be noted that the processor 150 may further control a magnitude of power supplied to the heating coil 130 in the on state. In the off state of the heating coil 130. the processor 150 may control the power supply 160 to not supply power to the heating coil 130.

In the active state of the air supply unit 140, the processor 150 may control the power supply 160 to supply power to the air supply unit 140. It may be noted that the processor 150 may further control a magnitude of power supplied to the air supply unit 140 in the active state. In the inactive state of the air supply unit 140, the processor 150 may control the power supply 160 to not supply power to the air supply unit 140.

In the illustrated embodiment of FIG. 3, the power supply 160 is electrically connected to each of the plurality of heating coils 130. The processor 150 may control the power supply 160 to independently supply power to the plurality of heating coils 130. Moreover, while not illustrated, the reading apparatus 110 may include separate power supplies 160 for the air supply unit 140 and the plurality of heating coils 130.

The reading apparatus 110 may further include a temperature sensor 135 (depicted schematically by a block in FIG. 3). The temperature sensor 135 may be communicably coupled to the processor 150. The temperature sensor 135 may be configured to sense the temperature of the heating coil 130. The temperature sensor 135 may be disposed proximal to the heating coil 130. The temperature sensor 135 may include, for example, a thermistor, a resistance temperature detector (RTD), a thermocouple, a diode, and the like.

The processor 150 may be further configured to determine the temperature of the heating coil 130 via the temperature sensor 135. The processor 150 may utilize the temperature sensor 135 to control the heating coil 130, such that the sterilization indicator 10 may achieve one or more preset temperatures in the closed-loop control system. The one or more preset temperatures may be temperatures for optimal growth of the spores 30 (shown in FIG. 1) in the substance 24.

Only one temperature sensor 135 is shown in FIG. 3 for illustrative purposes. It may be noted that the reading apparatus 110 may include a plurality of temperature sensors 135 corresponding to the plurality of heating coils 130. Each temperature sensor 135 from the plurality of temperature sensors 135 may be configured to sense the temperature of the corresponding heating coil 130. Further, the processor 150 may determine the temperature of the plurality of heating coils 130 via the plurality of temperature sensors 135.

The reading apparatus 110 may further include an excitation source (not shown) configured to excite the substance 24 (shown in FIG. 1) of the sterilization indicator 10. The excitation source may be any light source that causes the substance 24 of the sterilization indicator 10 to emit fluorescence. For example, the excitation source may emit ultraviolet (UV) electromagnetic radiation (having a wavelength ranging from 10 nanometers to 400 nanometers, preferably from 300 nanometers to 400 nanometers). The reading apparatus 110 may further include a sensor (not shown) configured to sense fluorescence emitted by the substance 24. The sensor may include a color sensor, an optical sensor, and the like. The sensor may generate one or more response signals upon sensing fluorescence emitted by the substance 24. Detection of fluorescence emitted by the substance 24 may be indicative of growth of spores 30 in the substance 24 or a positive result. The processor 150 may determine the fluorescence via the sensor, and display the positive result via the display 124 (shown in FIG. 2) of the reading apparatus 110.

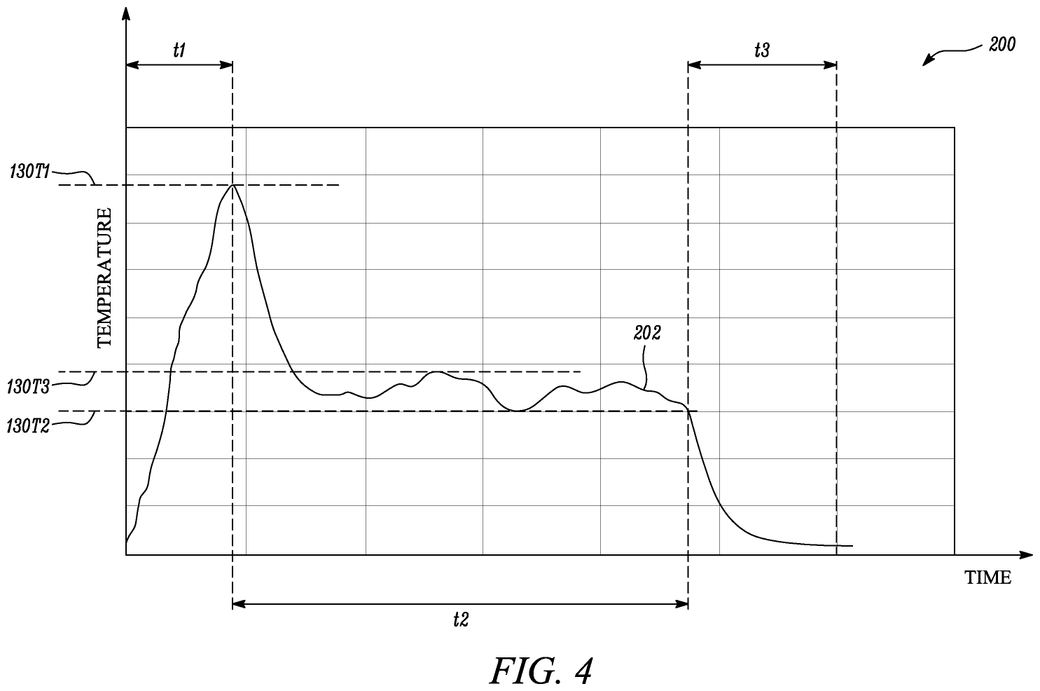

FIG. 4 illustrates a graph 200 depicting a variation of the temperature of the heating coil 130 with respect to time. Temperature is represented on an axis of ordinates (Y-axis) and time is represented on an axis of abscissas (X-axis). The graph 200 includes a curve 202 representing the temperature of the heating coil 130 with respect to time. The temperature of the heating coil 130 corresponds to the temperature sensed by the temperature sensor 135.

Referring to FIGS. 3 and 4, the processor 150 may be configured to control the heating coil 130 and the air supply unit 140, such that the heating coil 130 is in the on state and the temperature of the heating coil 130 rises to a first temperature 130T1 in a first time period t1. The air supply unit 140 may be maintained in the inactive state during the first time period t1. The processor 150 may control the heating coil 130 to achieve the first temperature 130T1 of the heating coil 130 in the first time period t1 based on the temperature determined via the temperature sensor 135.

The processor 150 may control the power supply 160 to supply power to the heating coil 130 to maintain the heating coil 130 in the on state during the first time period t1. The processor 150 may further control the power supply 160 to not supply power to the air supply unit 140 to maintain the air supply unit 140 in the inactive state during the first time period t1.

The heating coil 130 may radiatively heat the sterilization indicator 10 during the first time period t1 to increase the temperature of the substance 24 (shown in FIG. 1) to an optimal incubation temperature of the spores 30 (shown in FIG. 1) in the substance 24. A relation between the temperature of the heating coil 130 and the corresponding temperature of the substance 24 may be predetermined. For example, the processor 150 may utilize look-up-tables to determine the temperature of the heating coil 130 required to achieve a desired temperature of the substance 24. The relation between the temperature of the heating coil 130 and the corresponding temperature of the substance 24 may also take into consideration a thermal inertia of the substance 24.

The processor 150 may be further configured to control the heating coil 130 and the air supply unit 140. such that the heating coil 130 is in the on state and the temperature of the heating coil 130 is maintained between a second temperature 130T2 and a third temperature 130T3 during a second time period t2 subsequent to the first time period t1. The air supply unit 140 may be maintained in the active state during the second time period t2 in order to maintain the temperature of the heating coil 130 between the second temperature 130T2 and the third temperature 130T3. The second temperature 130T2 is less than the third temperature 130T3. Further, the third temperature 130T3 is less than the first temperature 130T1.

As depicted by the curve 202, as the air supply unit 140 is switched to the active state at the beginning of the second time period t2, the temperature of the heating coil 130 may drop significantly from the first temperature 130T1 to a temperature between the second temperature 130T2 and the third temperature 130T3.

The processor 150 may be configured to control the heating coil 130 and the air supply unit 140 to maintain the temperature of the heating coil 130 between the second temperature 130T2 and the third temperature 130T3 during the second time period t2 based on the temperature determined via the temperature sensor 135.

The processor 150 may control the power supply 160 to supply power to the heating coil 130 and the air supply unit 140 to maintain the heating coil 130 in the on state and the air supply unit 140 in the active state during the second time period t2.

In some embodiments, a difference between the second temperature 130T2 and the third temperature 130T3 may be less than or equal to 10% of the second temperature 130T2. For example, if the second temperature 130T2 is 55° C. the third temperature 130T3 may be between 60.5° C. (55° C.+5.5° C.) and 49.5° C. (55° C.−5.5° C.). In some embodiments, the difference between the second temperature 130T2 and the third temperature 130T3 may be less than or equal to 5% of the second temperature 130T2.

The heating coil 130 therefore may radiatively heat the sterilization indicator 10 during the second time period t2 to maintain the temperature of the substance 24 (shown in FIG. 1) around the optimal incubation temperature of the spores 30 (shown in FIG. 1) in the substance 24.

The processor 150 may determine a growth of the spores 30 (by detection of fluorescence by the sensor) during the second time period t2. The processor 150 may display the result of the sterilization of the sterilization indicator 10 via the display 124 (shown in FIG. 2). Thereafter, the processor 150 may cool down the heating coil 130.

Specifically, the processor 150 may be further configured to control the heating coil 130 and the air supply unit 140, such that the heating coil 130 is maintained in the off state during a third time period T3 subsequent to the second time period T2. The air supply unit 140 may be maintained in the active state during the third time period T3 in order to cool the heating coil 130. The processor 150 may control the power supply 160 to not supply power to the heating coil 130 to maintain the heating coil 130 in the off state during the third time period t3. The processor 150 may control the power supply 160 to supply power to the air supply unit 140 to maintain the air supply unit 140 in the active state during the third time period t3.

As depicted by the curve 202, the temperature of the heating coil 130 may drop significantly from between the second temperature 130T2 and the third temperature 130T3 during the third time period t3.

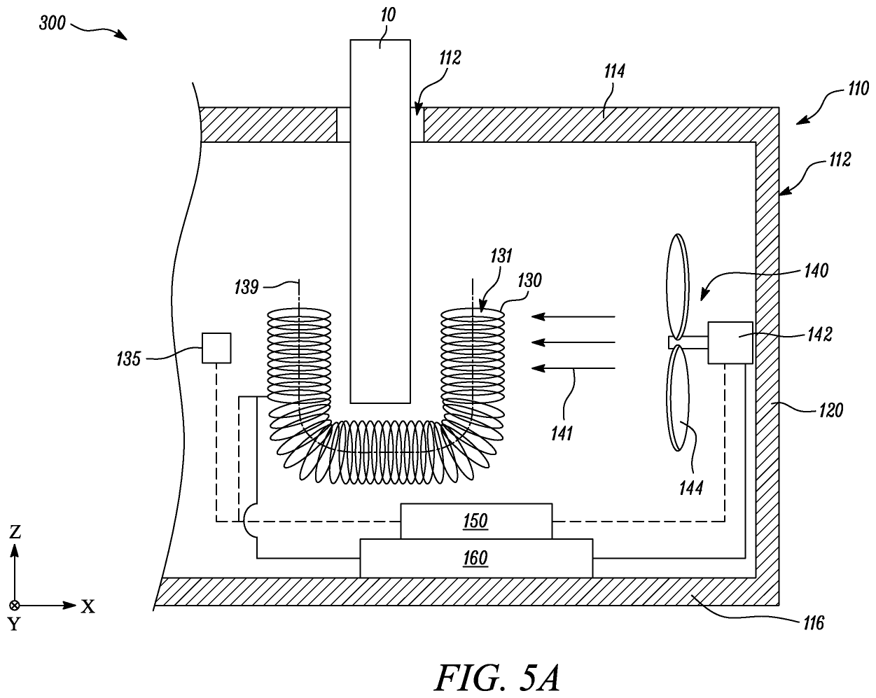

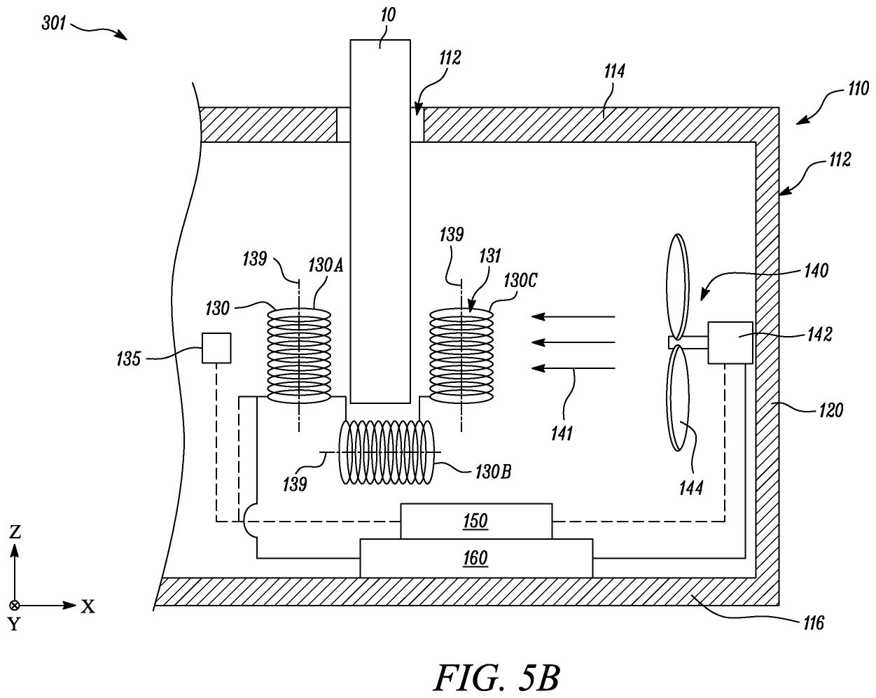

FIG. 5A illustrates a system 300 including the sterilization indicator 10 and the reading apparatus 110 according to another embodiment of the present disclosure. Specifically, FIG. 5A illustrates a portion of the reading apparatus 110 of the system 300. Some elements of the reading apparatus 110 are not shown in FIG. 5A for illustrative purposes.

The system 300 is similar to the system 100 of FIG. 3, with like elements designated by like reference characters. However, the heating coil 130 of FIG. 5A has a different configuration as compared to the heating coil 130 of FIG. 3.

Specifically, in the illustrated embodiment of FIG. 5A, the heating coil 130 has a U-shape. The coil axis 139 of the heating coil 130 may also have a U-shape. Upon insertion of the sterilization indicator 10 within the housing 112 through the aperture 122, the heating coil 130 may be disposed adjacent to the sterilization indicator 10, such that the sterilization indicator 10 is disposed outside the interior volume 131 of the heating coil 130. Further, upon insertion of the sterilization indicator 10 within the housing 112 through the aperture 122, the heating coil 130 may at least partially surround the sterilization indicator 10.

FIG. 5B illustrates a system 301 including the sterilization indicator 10 and the reading apparatus 110 according to another embodiment of the present disclosure. Specifically, FIG. 5B illustrates a portion of the reading apparatus 110 of the system 301. Some elements of the reading apparatus 110 are not shown in FIG. 5B for illustrative purposes.

The system 301 is substantially similar to the system 300 of FIG. 5A, with like elements designated by like reference characters. However, the heating coil 130 of FIG. 5B has a different configuration as compared to the heating coil 130 of FIG. 5A.

Specifically, in the illustrated embodiment of FIG. 5B, the heating coil 130 includes a plurality of heating coils 130A-130C spaced apart from each other. The plurality of heating coils 130A-130C may together form a U-shape. In the illustrated embodiment of FIG. 5B, the coil axis 139 of the heating coil 130A is substantially perpendicular to the coil axis 139 of the heating coil 130B. Furthermore, the coil axis 139 of the heating coil 130B is substantially perpendicular to the coil axis 139 of the heating coil 130C. Moreover, the coil axis 139 of the heating coil 130A is substantially parallel to the coil axis 139 of the heating coil 130C. The plurality of heating coils 130A-130C may at least partially surround the sterilization indicator 10. The plurality of heating coils 130A-130C may be electrically connected to each other in series.

While not illustrated in FIG. 5B, the reading apparatus 110 may include the plurality of apertures 122. The reading apparatus 110 may further include the plurality of heating coils 130A-130C corresponding to each of the plurality of apertures 122. Furthermore, in some cases, the plurality of heating coils 130A-130C corresponding to the plurality of apertures 122 may be electrically connected to each other in series. For example, one of the plurality of heating coils 130A-130C corresponding to one of the plurality of apertures 122 may be electrically connected in series with one (e.g., the adjacent one) of the plurality of heating coils 130A-130C corresponding to an adjacent aperture 122.

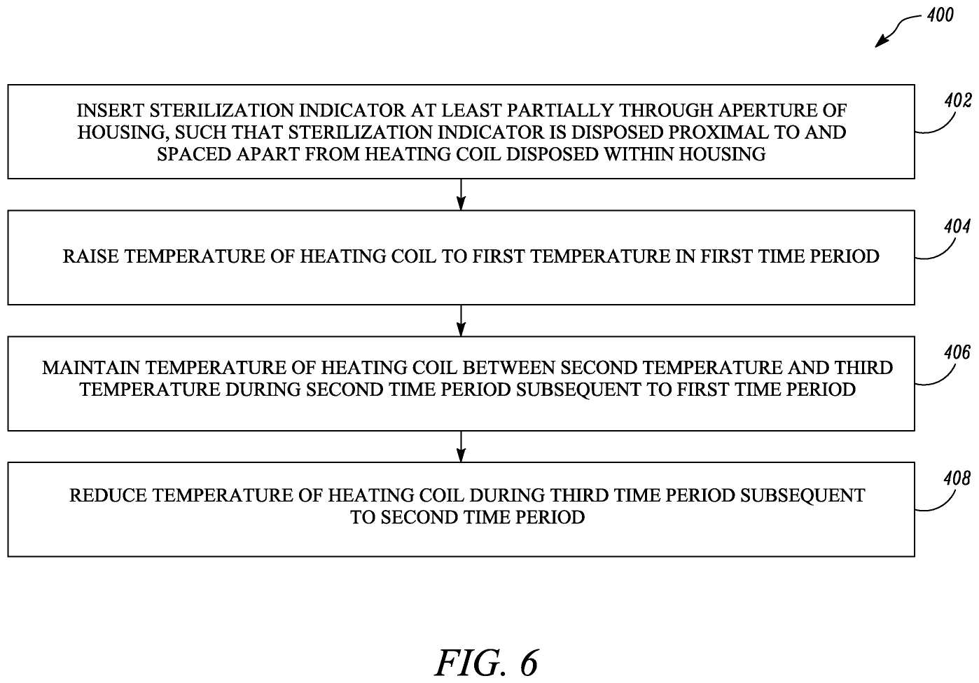

FIG. 6 illustrates a flowchart depicting various steps of a method 400 for incubating sterilization indicator having spores and a substance fluorescently responsive to a spore concentration according to an embodiment of the present disclosure. In some embodiments, the method 400 may be implemented by the processor 150 (shown in FIG. 3) of the reading apparatus 110. The method 400 will be further described with reference to FIGS. 1-3.

At step 402, the method 400 includes inserting the sterilization indicator at least partially through an aperture of a housing, such that the sterilization indicator is disposed proximal to and spaced apart from a heating coil disposed within the housing. The heating coil is configured to radiatively heat the sterilization indicator. For example, the method 400 may include inserting the sterilization indicator 10 at least partially through the aperture 122 of the housing 112, such that the sterilization indicator 10 is disposed proximal to and spaced apart from the heating coil 130 disposed within the housing 112.

At step 404, the method 400 further includes raising a temperature of the heating coil to a first temperature in a first time period by controlling, via a processor, power supply to the heating coil and the air supply unit during the first time period, such that the heating coil is an on state and the air supply unit is in an inactive state during first time period. In the on state, the heating coil is heated. In the inactive state, the air supply unit does not supply air to the heating coil.

For example, the method 400 may include raising the temperature of the heating coil 130 to the first temperature 130T1 in the first time period t1 by controlling, via the processor 150, power supply to the heating coil 130 and the air supply unit 140 during the first time period t1, such that the heating coil 130 is the on state and the air supply unit 140 is in the inactive state during first time period t1.

At step 406, the method 400 further includes maintaining the temperature of the heating coil between a second temperature and a third temperature during a second time period subsequent to the first time period by controlling, via the processor, power supply to the heating coil and the air supply unit during the second time period, such that the heating coil is maintained in the on state and the air supply unit is maintained in an active state during the second time period. In the active state, the air supply unit supplies air to the sterilization indicator.

For example, the method 400 may include maintaining the temperature of the heating coil 130 between the second temperature 130T2 and the third temperature 130T3 during the second time period t2 subsequent to the first time period t1 by controlling, via the processor 150, power supply to the heating coil 130 and the air supply unit 140 during the second time period t2, such that the heating coil 130 is maintained in the on state and the air supply unit 140 is maintained in the active state during the second time period t2.

At step 408, the method 400 further includes reducing the temperature of the heating coil during a third time period subsequent to the second time period by controlling, via the processor, power supply to the heating coil and the air supply unit, such that the heating coil is maintained in an off state and the air supply unit is maintained in the active state during the third time period. In the off state, the heating coil is not heated.

For example, the method 400 may include reducing the temperature of the heating coil 130 during the third time period t3 subsequent to the second time period t2 by controlling, via the processor 150, power supply to the heating coil 130 and the air supply unit 140, such that the heating coil 130 is maintained in the off state and the air supply unit 140 is maintained in the active state during the third time period t3.

In some embodiments, the method 400 further includes determining the temperature of the heating coil via a temperature sensor. In some embodiments, the method 400 further includes controlling, via the processor, power supply to the heating coil based on the temperature of the heating coil. In some embodiments, the method 400 further includes controlling, via the processor, power supply to the air supply unit based on the temperature of the heating coil.

For example, the method 400 may include: determining the temperature of the heating coil 130 via the temperature sensor 135; controlling, via the processor 150, power supply to the heating coil 130 based on the temperature of the heating coil 130; and controlling, via the processor 150, power supply to the air supply unit 140 based on the temperature of the heating coil 130.

The method 400 may allow rapidly heating the sterilization indicator 10 from room temperature to its optimal temperature, and maintaining the optimal temperature throughout incubation, thereby decreasing the time to result of the reading apparatus 110.

### Experimental Results

Experiments were conducted on the reading apparatus 110 of the present disclosure. Referring to FIGS. 1-3, the sterilization indicator 10 was inserted into the housing 112 through the aperture 122 of the reading apparatus 110. The sterilization indicator 10 was activated prior to being inserted into the housing 112. The sterilization indicator 10 was incubated by the reading apparatus 110. A thermocouple was inserted into the sterilization indicator 10 to determine a temperature of the substance 24 of the sterilization indicator 10.

The heater coil 130 used in the experiments was made from nickel-chromium. The air supply unit 160 had an airflow of 23 cubic feet per minute (CFM) at 24 Volts. The power supply 160 was an adjustable DC power supply. The experiments were conducted at an ambient temperature of about 23° C. The results were determined and were plotted on graphs that are described hereinafter with further reference to FIGS. 1-3.

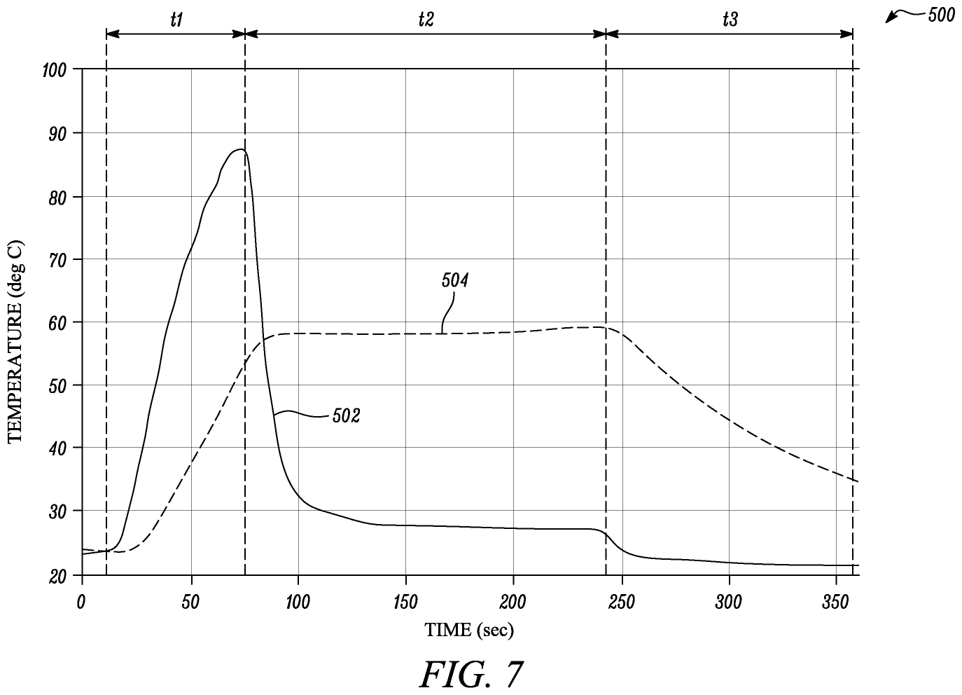

FIG. 7 illustrates a graph 500 representing temperature (in ° C.) on an axis of ordinates (Y-axis) and time (in seconds) on an axis of abscissas (X-axis). The graph 500 includes a first curve 502 depicting the temperature of the heating coil 130 as measured by the temperature sensor 135. The graph 500 further includes a second curve 504 depicting the temperature of the substance 24 of the sterilization indicator 10.

The heating coil 130 was supplied with 15 volts, 1.9 amperes (i.e., 28.5 Watts) via the power supply 160, and the air supply unit 140 was maintained at the inactive state during the first time period t1 (about 45 seconds). In this experiment, the optimal incubation temperature of the spores 30 was about 60 degrees.

As depicted by the first curve 502, the temperature of the heating coil 130 was raised to about 87° C. during the first time period t1. As depicted by the second curve 504, the temperature of the substance 24 rose to about 59° C, during the first time period t1 due to the rise of the temperature of the heating coil 130.

Further, as depicted by the first curve 502, the temperature of the heating coil 130 was maintained between about 30° C. and about 25° C. during the second time period t2 (about 170 seconds). The air supply unit 140 was maintained at the active state during the second time period t2, such that the temperature of the heating coil 130 was maintained between about 30° C. and about 25° C. during the second time period t2.

As depicted by the second curve 504, maintaining the temperature of the heating coil 130 between about 25° C. and about 30° C. during the second time period t2 caused the temperature of the substance 24 to be maintained at about 59° C. during the second time period t2. The air supply unit 140 was maintained in the active state during the second time period t2 to maintain the temperature of the heating coil 130 between about 25° C. and about 30° C. during the second time period t2. The growth of the spores 30 was determined during the second time period t2.

As depicted by the first curve 502, the power supply to the heating coil 130 was discontinued during the third time interval t3. The air supply unit 140 was maintained at the active state during the third time period t3 to cool down the heating coil 130.

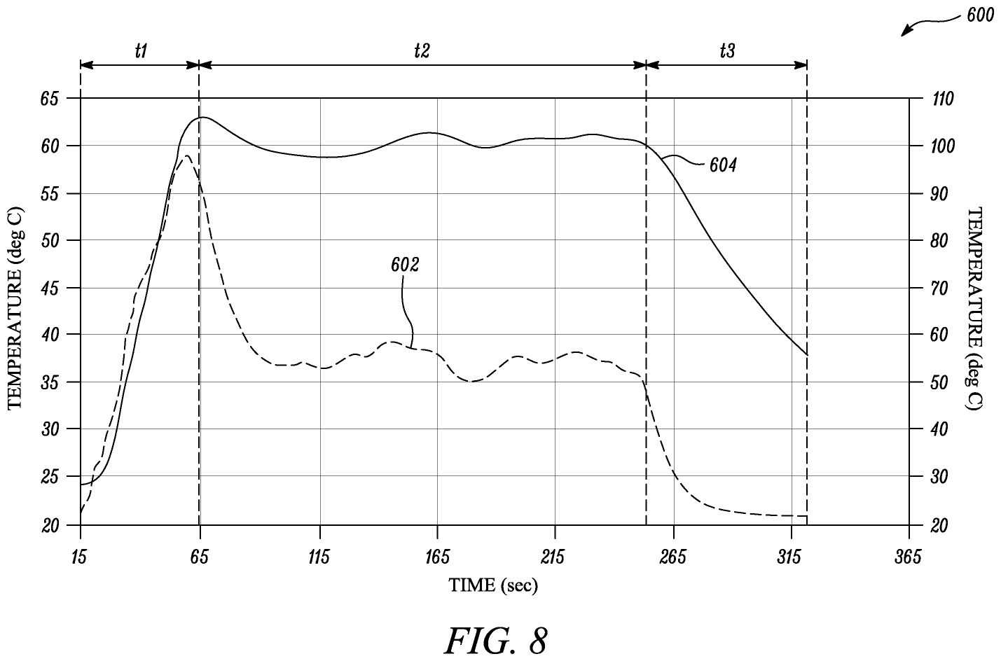

FIG. 8 illustrates a graph 600 representing temperature (in ° C.) on an axis of ordinates (Y-axis) and time (in seconds) on an axis of abscissas (X-axis). The left Y-axis represents the temperature of the temperature of the substance 24 of the sterilization indicator 10, and the right Y-axis represents the temperature of the heating coil 130 as measured by the temperature sensor 135. The graph 600 includes a first curve 602 depicting the temperature of the heating coil 130 as measured by the temperature sensor 135. The graph 600 further includes a second curve 604 depicting the temperature of the substance 24 of the sterilization indicator 10.

The heating coil 130 was supplied with 60 Watts via the power supply 160, and the air supply unit 140 was maintained at the inactive state during the first time period t1 (about 40 seconds). In this experiment, the optimal incubation temperature of the spores 30 was about 60 degrees.

As depicted by the first curve 602, the temperature of the heating coil 130 was raised to about 96° C. during the first time period t1. As depicted by the second curve 604, the temperature of the substance 24 rose to about 63° C. during the first time period t1 due to the rise of the temperature of the heating coil 130.

Further, as depicted by the first curve 602, the temperature of the heating coil 130 was maintained between about 50° C. and about 60° C. during the second time period t2 (about 190 seconds). The air supply unit 140 was maintained at the active state during the second time period t2, such that the temperature of the heating coil 130 was maintained between about 50° C. and about 60° C. during the second time period t2.

As depicted by the second curve 604, maintaining the temperature of the heating coil 130 between about 50° C. and about 50° C. during the second time period t2 caused the temperature of the substance 24 to be maintained at about 60° C. during the second time period t2. The air supply unit 140 was maintained in the active state during the second time period t2 to maintain the temperature of the heating coil 130 between about 50° C. and about 60° C. during the second time period t2. The growth of the spores 30 was determined during the second time period t2.

As depicted by the first curve 602, the power supply to the heating coil 130 was discontinued during the third time interval t3. The air supply unit 140 was maintained at the active state during the third time period t3 to cool down the heating coil 130.

The experiment was reconducted multiple times with a variac selected as the power supply 160. Voltage and current were adjusted using the variac. The temperature of the substance 24 at 60 seconds, and the time taken by the substance 24 to reach 56° C. was observed. The results are provided in Table 1 below.

TABLE 1
Results of the Experiment

| | | | | Time taken | Heating | Heating |
|---|---|---|---|---|---|---|
| Volt- | Cur- | | Temperature | by Substance | Rate | Rate |
| age | rent | Power | of Substance | to reach | (deg/ | (deg/ |
| (V) | (A) | (W) | (at 60 secs) | 56° C. (sec) | sec) | min) |
| 11.2 | 2.6 | 29 | 21.329 | 189 | 0.184 | 11.03 |
| 15 | 3.49 | 52 | 23.194 | 83 | 0.4 | 24 |
| 18 | 4.19 | 75 | 23.854 | 48 | 0.677 | 40.65 |
| 20 | 4.65 | 93 | 23.233 | 44 | 0.758 | 45.49 |
| 24 | 5.58 | 134 | 23.57 | 32 | 1.038 | 62.25 |
| 30 | 6.98 | 209 | 23.297 | 24 | 1.398 | 83.91 |
| 34 | 7.91 | 269 | 24.131 | 18 | 1.928 | 115.71 |

It was concluded that the reading apparatus 110 facilitated rapid heating of the sterilization indicator 10 from room temperature to its optimal temperature, and maintaining the optimal temperature throughout incubation, thereby decreasing the time to result of the reading apparatus 110.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A sterilization indicator reading apparatus for use with a sterilization indicator having spores and a substance fluorescently responsive to a spore concentration, the sterilization indicator reading apparatus comprising:
a housing comprising a top portion and a bottom portion opposite the top portion, the top portion defining an aperture therethrough, wherein the aperture is dimensioned to at least partially receive the sterilization indicator therethrough, such that the sterilization indicator is at least partially received within the housing;
a heating coil disposed within the housing between the top portion and the bottom portion, wherein the heating coil is at least partially aligned with the aperture and defines an interior volume, wherein, upon insertion of the sterilization indicator into the housing through the aperture, the sterilization indicator is at least partially received in the interior volume of the heating coil, and wherein the heating coil is spaced apart from the sterilization indicator when the sterilization indicator is at least partially received in the interior volume of the heating coil and the heating coil is configured to radiatively heat the sterilization indicator, wherein the heating coil is configured to be switched between an on state and an off state, wherein, in the on state, the heating coil is heated, and wherein, in the off state, the heating coil is not heated; and
an air supply unit disposed within the housing and configured to selectively supply air to the heating coil, wherein the air supply unit is configured to be switched between an active state and an inactive state, wherein, in the active state, the air supply unit supplies air to the heating coil, and wherein, in the inactive state, the air supply unit does not supply air to the heating coil.

2. The sterilization indicator reading apparatus of claim 1, wherein the heating coil comprises nickel-chromium.

3. The sterilization indicator reading apparatus of claim 1, further comprising a processor communicably coupled to the heating coil and the air supply unit, wherein the processor is configured to control the heating coil and the air supply unit, such that:
the heating coil is in the on state and a temperature of the heating coil rises to a first temperature in a first time period, wherein the air supply unit is maintained in the inactive state during the first time period;
the heating coil in the on state and the temperature of the heating coil is maintained between a second temperature and a third temperature during a second time period subsequent to the first time period, wherein the air supply unit is maintained in the active state during the second time period in order to maintain the temperature of the heating coil between the second temperature and the third temperature; and
the heating coil is maintained in the on state during a third time period subsequent to the second time period, wherein the air supply unit is maintained in the active state during the third time period to cool the heating coil.

4. The sterilization indicator reading apparatus of claim 3, further comprising a temperature sensor communicably coupled to the processor and configured to sense the temperature of the heating coil, wherein the processor is further configured to:
determine the temperature of the heating coil via the temperature sensor;
control the heating coil to achieve the first temperature of the heating coil in the first time period based on the temperature determined via the temperature sensor; and
control the heating coil and the air supply unit to maintain the temperature of the heating coil between the second temperature and the third temperature during the second time period based on the temperature determined via the temperature sensor.

5. The sterilization indicator reading apparatus of claim 3, wherein a difference between the second temperature and the third temperature is less than or equal to 10% of the second temperature.

6. The sterilization indicator reading apparatus of claim 3, further comprising a power supply communicably coupled to the processor and electrically connected to the heating coil and the air supply unit, wherein:
in the on state of the heating coil, the processor controls the power supply to supply power to the heating coil;
in the off state of the heating coil, the processor controls the power supply to not supply power to the heating coil;
in the active state of the air supply unit, the processor controls the power supply to supply power to the air supply unit; and
in the inactive state of the air supply unit, the processor controls the power supply to not supply power to the air supply unit.

7. The sterilization indicator reading apparatus of claim 6, wherein the power supply comprises a variac.

8. The sterilization indicator reading apparatus of claim 1, wherein the aperture comprises a plurality of apertures spaced apart from each other, wherein the heating coil comprises a plurality of heating coils corresponding to the plurality of apertures, wherein the plurality of apertures and the plurality of heating coils are configured to removably receive a corresponding plurality of sterilization indicators, and wherein the air supply unit is configured to supply air to the plurality of heating coils.

9. The sterilization indicator reading apparatus of claim 1, wherein the air supply unit comprises at least one fan.

10. A sterilization indicator reading apparatus for use with a sterilization indicator having spores and a substance fluorescently responsive to a spore concentration, the sterilization indicator reading apparatus comprising:
a housing comprising a top portion, and a bottom portion opposite the top portion, the top portion defining an aperture therethrough, wherein the aperture is dimensioned to at least partially receive the sterilization indicator therethrough, such that the sterilization indicator is at least partially received within the housing;
a heating coil disposed within the housing between the top portion and the bottom portion, wherein, upon insertion of the sterilization indicator within the housing through the aperture, the sterilization indicator is disposed proximal to and spaced apart from the heating coil, such that the heating coil is configured to radiatively heat the sterilization indicator, wherein the heating coil is configured to be switched between an on state and an off state, wherein, in the on state, the heating coil is heated, and wherein, in the off state, the heating coil is not heated;
an air supply unit disposed within the housing and configured to selectively supply air to the heating coil, wherein the air supply unit is configured to be switched between an active state and an inactive state, wherein, in the active state, the air supply unit supplies air to the heating coil, and wherein, in the inactive state, the air supply unit does not supply air to the heating coil; and
a processor communicably coupled to the heating coil and the air supply unit, wherein the processor is configured to control the heating coil and the air supply unit, such that:
the heating coil is in the on state and a temperature of the heating coil rises to a first temperature in a first time period, wherein the air supply unit is maintained in the inactive state during the first time period;
the heating coil is in the on state and the temperature of the heating coil is maintained between a second temperature and a third temperature during a second time period subsequent to the first time period, wherein the air supply unit is maintained in the active state during the second time period in order to maintain the temperature of the heating coil between the second temperature and the third temperature; and
the heating coil is maintained in the off state during a third time period subsequent to the second time period, wherein the air supply unit is maintained in the active state during the third time period in order to cool the heating coil.

11. The sterilization indicator reading apparatus of claim 10, wherein the heating coil comprises nickel-chromium.

12. The sterilization indicator reading apparatus of claim 10, further comprising a temperature sensor communicably coupled to the processor and configured to sense the temperature of the heating coil, wherein the processor is further configured to:
determine the temperature of the heating coil via the temperature sensor;
control the heating coil to achieve the first temperature of the heating coil in the first time period based on the temperature determined via the temperature sensor; and
control the heating coil and the air supply unit to maintain the temperature of the heating coil between the second temperature and the third temperature during the second time period based on the temperature determined via the temperature sensor.

13. The sterilization indicator reading apparatus of claim 10, wherein a difference between the second temperature and the third temperature is less than or equal to 10% of the second temperature.

14. The sterilization indicator reading apparatus of claim 10, further comprising a power supply communicably coupled to the processor and electrically connected to the heating coil and the air supply unit, wherein:
in the on state of the heating coil, the processor controls the power supply to supply power to the heating coil;
in the off state of the heating coil, the processor controls the power supply to not supply power to the heating coil;
in the active state of the air supply unit, the processor controls the power supply to supply power to the air supply unit; and
in the inactive state of the air supply unit, the processor controls the power supply to not supply power to the air supply unit.

15. The sterilization indicator reading apparatus of claim 14, wherein the power supply comprises a variac.

16. The sterilization indicator reading apparatus of claim 10, wherein the heating coil is at least partially aligned with the aperture and defines an interior volume therein, wherein, upon insertion of the sterilization indicator within the housing through the aperture, the sterilization indicator is at least partially received in the interior volume of the heating coil, wherein the heating coil is dimensioned, such that the heating coil is spaced apart from the sterilization indicator when the sterilization indicator is at least partially received in the interior volume of the heating coil.

17. The sterilization indicator reading apparatus of claim 10, wherein the heating coil defines an interior volume therein, and wherein, upon insertion of the sterilization indicator within the housing through the aperture, the heating coil is disposed adjacent to the sterilization indicator, such that the sterilization indicator is disposed outside the interior volume of the heating coil.

18. The sterilization indicator reading apparatus of claim 17, wherein the heating coil comprises a plurality of heating coils spaced apart from each other and at least partially surrounding the sterilization indicator.

19. A method for incubating a sterilization indicator having spores and a substance fluorescently responsive to a spore concentration, the method comprising:
inserting the sterilization indicator at least partially through an aperture of a housing, such that the sterilization indicator is disposed proximal to and spaced apart from a heating coil disposed within the housing, wherein the heating coil is configured to radiatively heat the sterilization indicator;
raising a temperature of the heating coil to a first temperature in a first time period by controlling, via a processor, power supply to the heating coil and an air supply unit during the first time period, such that the heating coil is an on state and the air supply unit is in an inactive state during first time period, wherein, in the on state, the heating coil is heated, and wherein, in the inactive state, the air supply unit does not supply air to the heating coil;
maintaining the temperature of the heating coil between a second temperature and a third temperature during a second time period subsequent to the first time period by controlling, via the processor, power supply to the heating coil and the air supply unit during the second time period, such that the heating coil is maintained in the on state and the air supply unit is maintained in an active state during the second time period, wherein in the active state, the air supply unit supplies air to the sterilization indicator; and
reducing the temperature of the heating coil during a third time period subsequent to the second time period by controlling, via the processor, power supply to the heating coil and the air supply unit, such that the heating coil is maintained in an off state and the air supply unit is maintained in the active state during the third time period, wherein, in the off state, the heating coil is not heated.

20. The method of claim 19, further comprising:
determining the temperature of the heating coil via a temperature sensor;
controlling, via the processor, power supply to the heating coil based on the temperature of the heating coil; and
controlling, via the processor, power supply to the air supply unit based on the temperature of the heating coil.
